(19) United States
(12) Patent Application Publication
(10) Pub. No.: US 2009/0325268 A1
(43) Pub. Date: Dec. 31, 2009

(54) PROLINE-SPECIFIC PROTEASES FREE FROM AMYLOLYTIC ACTIVITIES

(76) Inventors: Luppo Edens, Rotterdam (NL); Rudolf Franciscus WIlhelmus Cornelis Van Beckhoven, Bavel (NL); Michael Dennis Tabeling, Delft (NL)

Correspondence Address: NIXON & VANDERHYE, PC, 901 NORTH GLEBE ROAD, 11TH FLOOR, ARLINGTON, VA, 22203, US

(21) Appl. No.: 12/298,076

(22) PCT Filed: Apr. 23, 2007
(86) PCT No.: PCT/EP07/53940

§ 371 Date: Jun. 2, 2009

(30) Foreign Application Priority Data
Apr. 25, 2006 (EP) 06113062.1

(51) Int. Cl. C12N 9/48

(52) U.S. Cl. 435/212

(57) ABSTRACT

The invention relates to a proline-specific protease preparation free from amylolytic activity and a purification method for obtaining the enzyme preparation according to the invention.

The invention relates to a process for the production of a proline-specific protease preparation which is substantially free from contaminating amylolytic side activities, the proline-specific protease preparation thus obtained and uses thereof.

Proline-specific proteases form an emerging class of industrial enzymes with advantageous properties for the production of protein hydrolysates as well as in preventing haze formation in various liquid food products. For example, in WO 02/45523 a substantial debittering of hydrolysates produced from proline-rich protein substrates by proline-specific proteases is described. Protein hydrolysates are frequently used in infant formula and clinical nutrition. Aim of these products is to prevent the development of protein allergenicity and to ensure an efficient metabolisation of the protein supplied. Protein hydrolysates in products destined for consumers with non-medical needs, for example athletes or people on a slimming diet, must be tailored to provide good taste characteristics. Most of such protein hydrolysates are obtained from cow milk proteins, almost exclusively from the whey protein fraction of cow milk. The popularity of whey proteins for this purpose is not only based on the fact that in many countries whey proteins are cheaper than the caseins, but also because upon enzymatic hydrolysis casein becomes notoriously bitter in contrast to whey protein. Because 80 percent of the proteins present in cow milk are caseins, it seems safe to assume that these caseins fullfil a nutritional role that is not met by the whey fraction of milk. Therefore, making casein hydrolysates available for human consumption has a considerable nutritional and thus economical relevance.

Another useful application of proline-specific proteases is described in WO 02/046381. In this application an enzymatic method is described by which haze formation in beer, wine and fruit juices can be prevented. The haze formed in these beverages usually represents a colloidal precipitate of aggregates between proline rich (“haze active”) proteins and plant polyphenols. During the production of beer, wines and fruit juices, both proteins and polyphenols are extracted from the plant and/or fruit tissue disrupted during the initial production phase.

In beer production, the haze active proteins as well as a major part of the polyphenols, are extracted from the malted barley. The resulting protein-polyphenol precipitate that develops during beer fermentation and maturation, may eventually lead to a socalled “chill haze” in bottled beer. The prevention of this chill haze formation in beer is a technically difficult and expensive process for which conventionally polyvinylpolypyrrolidone (PVPP) or silica hydrogel treatments are being used. In contrast with the convential methods, the enzymatic chill haze prevention approach according to WO 02/046381, is relatively cheap and simple. In the production of beer the proline-specific protease is added during the fermentation step hereby minimizing the risk of beer oxidation. Long incubation times (during the whole fermentation phase) guarantee that minimal levels of the proline-specific protease suffice to degrade all haze active proteins. Due to these advantages and taking the huge production volumes of beer into account, the cost savings that can be obtained by this enzymatic method are considerable.

We have now found that upon using a proline-specific protease in any of the aforementioned applications, it is of paramount importance that the proline-specific protease preferably is devoid of amylolytic side activities. Too high levels of these amylolytic side activities in the proline-specific protease preparation, may have detrimental side-effects as a result of decomposition of polysaccharides present in the relevant final products. This decomposition of the polysaccharide fraction may lead to, for example, a softening or even liquefaction of solid end products or an impaired mouthfeel of a beverage such as beer. It is therefore an object of the present invention to provide a proline-specific protease preparation which is substantially free from contaminating amylolytic side activities.

With the term “proline-specific protease” is meant any endoprotease or exopeptidase which is capable of cleaving a peptide bond involving a proline residue. That is to say, the a “proline-specific protease” is one which cleaves a peptide or protein at a position where the peptide or protein comprises a proline residue. Examples of endoproteases capable of cleaving such peptide bonds are prolyl oligopeptidases (EC 3.4.21.26; Fülop et al., Cell 1998, 94, 161-170) as well as the endoproteases belonging to the S28 family of clan SC of serine proteases (Edens et al., J Agric Food Chem 53: 7950-7957, 2005; Handbook of Proteolytic Enzymes; Barrett A. J.; Rawlings N. D.; Woessner J. F., Eds.; Academic Press, London, UK, 1998, 369-415). Among the exoproteases capable of cleaving peptide bonds involving proline residues the enzymes dipeptidyl peptidase IV (EC 3.4.14.4) and dipeptidyl peptidase II EC 3.4.14.2) are worth mentioning.

With the term “amylolytic side activities” is meant any enzymatic activity that can hydrolyse 1,4-alpha-D-glucosidic linkages in polysaccharides such as starch, dextrins, glycogen etceteras. Therefore, the term “amylolytic side activities” comprises enzymes such as α-amylase (EC 3.2.1.1), β-amylase (EC 3.2.1.2), glucoamylase (EC 3.2.1.3), glucan 1,4-alpha-maltohydrolase (EC 3.2.1.133) as well as mixtures of these activities.

By the term “substiantially free from amylolytic side activities” is meant that the amylolytic side activities are present at such a low level that, upon effective dosage of the proline-specific protase activity in the respective production process, no observable decomposition of poly- and oligosaccharides with the associated negative effects as described above occurs in said production process. This means that the allowable level of contaminating amylolytic side activities in the proline-specific proteases may vary from production process to production process, depending on the particular process conditions as well as on the level and type of polysaccharides present.

The term “substiantially free from amylolytic side activities” may also be expressed as a ratio of the activity of a given amylolytic side activity (in certain units) divided by the activity of a given proline-specific protease activity (in certain units). This ratio may vary from production process to production process and will depend on the particular proline-specific protease used in the production process, as well as the particular amylolytic side activity that is present in the particular proline-specific protease used.

Advantages of the separation method according to the invention is, that with the method of the invention the yield of the proline-specific protease is at least 65%, preferably at least 75%, while the residual amylolytic activity, for example alpha-amylolytic activity, is generally 5.0 or lower FAU per PPU, such as 0.5 or lower FAU per PPU, for example 0.05 or lower FAU per PPU, such as 0.005 or lower FAU per PPU, preferably 0.0005 or lower FAU/PPU. The amyloglucosidase activity will generally be 1 or less AGI per PPU, for example 0.5 or less AGI for PPU, such as 0.1 AGI per PPU, or 0.01 or less AGI per PPU. The definitions of FAU and PPU are specified in the Materials & Methods section of this application. Also, the definition for glucoamylase activities (“AGI”) is provided in that section.

In the event that the proline-specific protease has a pH optimum higher than 5.5, the PPU measurement is typically carried out at pH 6.5, for example at 37° C., instead of 4.6 and 37° C.

In a first aspect, the invention provides a process for the production of a proline-specific protease that is substantially free from amylolytic side activities, comprising one or more liquid chromatographic separation steps, preferably a single chromatographic separation step. Many different chromatographic separation methods are known in the art and these can be screened in order to provide the proline-specific protease of the present invention. Suitable chromatographic separation methods comprise ion exchange chromatography, affinity chromatography, size exclusion chromatography, hydrophobic interaction chromatography and others. For the present invention preferably ion exchange chromatography and/or hydrophobic interaction chromatography are used.

The proline-specific protease of interest may be produced by fermentation processes using microorganisms such as fungi, yeast and bacteria, that produce and preferably secrete the proline-specific protease of interest in the fermentation broth. In the art, such fermentation processes are known, see for example WO 02/45524. In the processes of the prior art, the proline-specific protease may be recovered from the fermentation broth by techniques also known in the art. As a first step, the cells of the production organism may be separated from the broth by centrifugation or filtration.

In case the proline-specific protease is secreted by the microorganism in the broth, the cell free broth may be concentrated, for example by ultrafiltration, and the proline-specific protease preparation thus obtained may be stabilized by known stabilizers such as glycerol or other polyols.

In case the proline-specific protease is not secreted by the microorganism but remains intracellular, the production organism has to be lysed to release the relevant proline-specific protease activity. After another filtration or centrifugation step to remove the cell debris, the liquid fraction may be concentrated and stabilized as described above for the excreted proline-specific protease. A solid preparation may be obtained from the optionally concentrated proline-specific protease solutions by known precipitation and/or evaporation and/or (spray) drying techniques.

According to the process of the present invention, the cell free broth or cell debris free liquid fraction, may be subjected to one or more chromatographic separation steps in order to provide the proline-specific protease preparation of the invention that is substantially free from amylolytic side activities. Preferably, such a chromatographic separation step is carried out on preparations that have not yet been stabilized by glycerol or polyol additions. Selecting the most appropriate chromatographic separation methods is highly dependent on, for example, the molecular characteristics of the proline-specific protease and the contaminating amylolytic activities. Relevant characteristics include isoelectric point, hydrophobicity, molecular surface charge distribution, molecular weight and several other protein chemical properties. A practical background on the use of these characteristics in enzyme purification can be found in a.o. the Protein Purification Handbook (issued by Amersham Pharmacia Biotech, nowadays GE Healthcare Bio-Sciences, Diegem, Belgium).

However, the chromatographic separation step will be considerably more complicated in those cases in which the proline-specific protease is not secreted by the microorganisms or if the amylolytic side activities exhibit molecular characteristics which are very similar to molecular characteristics of the proline-specific protease. For example, similar isoelectric points present a typical complication in for instance ion exchange chromatography and whereby the producing microorganism secretes a large variety of enzymes, one of which is the proline-specific protease. In these cases the purification of the desired proline-specific protease from the contaminating enzymes is not trivial, certainly not if this purification has to take place cost-effectively and on a large, industrial scale. After an extensive investigation of a large variety of chromatographic resins and elution protocols, we have been able to devise an industrially applicable, one-column separation protocol resulting in the complete separation of proteolytic and multiple amylolytic activities typically having very similar isoelectric points. Preferred purification methods for the proline-specific endoprotease from A. niger according to the invention make use of ion-exchange or hydrophobic interaction chromatography. These purification methods are exemplified in Example 2 of the present application.

In a second aspect, the present invention provides a proline-specific protease that is substantially free from amylolytic side activities. Such proline-specific proteases can advantageously be used in those applications wherein the decomposition of polysaccharides is undesired or unwanted. As some amylolytic activities are relatively heat stable, they tend to survive the commonly used enzyme inactivation protocols or product pasteurization protocols. As a result protein hydrolysates produced using proline-specific proteases may contain traces of residual amylolytic activities causing serious problems if formulated in combination with poly- or oligosaccharides. Thus, the proline-specific protease obtainable by a method of the invention is preferably used for all products in which the resulting protein hydrolysate is combined with starch or maltodextrin containing compounds. Preferably this product is solid, e.g. an energy or protein bar, a powder, e.g. a powder to be incorporated in an infant formula or a powder to prepare nutrient mixtures in the form of a shake, or the product can be a liquid, such as a liquid infant formula or a power drink.

Furthermore, the proline-specific protease obtainable by a method of the invention can advantageously be used in a final product containing high amounts of the active enzyme. Such final products have been described in, inter alia, WO 2005/027953 and our pending application PCT/EP2007/000896. These final products are consumed together with products that may contain wheat gluten with the intention to minimize the effect of toxic gluten epitopes and are of particular relevance for people intolerant to gluten, such as celiac patients.

Finally, the proline-specific protease obtainable by a method of the invention can be used in haze prevention in all plant derived liquid products. Preferably this plant derived liquid product is beer. In the latter application use of the proline-specific protease obtainable by a method of the invention prevents an over degradation of poly- and oligosaccharides thus leading to an improved mouthfeel of the final beer.

### Materials and Methods

### Proline-Specific Endo Proteolytic Activity

A. niger derived proline specific endoproteolytic activity was tested using CBZ-Gly-Pro-pNA (Bachem, Bubendorf, Switzerland) as a substrate at 37° C. in a citrate/disodium phosphate buffer pH 4.6. The reaction products were monitored spectrophotometrically at 405 nM. The increase in absorbance at 405 nm in time is a measure for enzyme activity. A Proline Protease Unit (PPU) is defined as the quantity of enzyme that releases 1 μmol of p-nitroanilide per minute under the conditions specified and at a substrate concentration of 0.37 mM Z-Gly-Pro-pNA.

### Alpha-Amylase Activity

The alpha-amylase measuring method used is based upon the Ceralpha test kit as supplied by Megazyme (R-CAAR-4; Megazyme International Ireland Ltd., Bray, Ireland). In this method, the sample is incubated with a ‘non-reducing and end-blocked p-nitrophenylmaltoheptaoside’ (BNPG7) plus excess levels of amyloglucosidase and alpha-glucosidase. Upon the hydrolysis of this oligosaccharide by an endo-acting alpha-amylase, the excess quantities of amyloglucosidase and alpha-glucosidase present in the mixture, lead to an instantaneous and quantitative hydrolysis of the p-nitrophenol-linked substrate. In the incubation, 90 microL substrate solution reacts with 10 microL sample solution, containing between 0.001 and 0.01 FAU per ml. After 425 seconds of incubation (at pH 5.2 and 37° C.), the incubation reaction is terminated and the colour is developed by adding 75 microL alkaline (20.5 g/l) TRIS solution. The increase in colour at 405 nm is proportional to the amylolytic enzyme activity present in the sample. For this method an alpha amylase containing standard preparation from Aspergillus oryzae was used for calibration of the system. The activity of this standard is expressed in FAU (Fungal Amylase Units). One FAU is defined as the amount of enzyme that converts one gram of soluble starch per hour in a product having an equal absorption to a reference colour at 620 nm after reaction with iodine at pH 5.0 and 30° C. and a reaction time between 15 and 25 minutes. The reference colour is defined as the absorbance of a CoCl₂ colour standard consisting of 25.0 g CoCl₂.H₂O and 3.84 potassium dichromate in 100 ml of 0.01 N hydrochloric acid. Accordingly, an absorbance increase of 0.54 at 405 nm corresponds to an amylase activity of 0.005 FAU per ml. The measuring range of the method is from 0.001 to 0.01 FAU per ml which corresponds to an increase of absorbance between 0.11 and 1.1.

### Amyloglucosidase Activity

To quantify amylolytic exo-activity in the samples, amyloglucosidase activities were measured. The amyloglucosidase assay reagent (R-AMGR3) as supplied by Megazyme International Ireland Ltd was used. Amyloglucosidase activity is determined at 37° C. and pH 4.50 using p-nitrophenyl-β-maltoside as the substrate. One AmyloGlucosidase Unit (AGI) is defined as the amount of enzyme that produces 1 μmol of glucose per minute from soluble starch at pH 4.3 and 60° C. Enzymatic hydrolysis of p-nitrophenyl-β-maltoside results in the release of p-nitrophenol and cellobiose. The presence of excess β-glucosidase, added via the reagent, ensures hydrolysis of cellobiose to glucose, preventing competitive inhibition of amyloglucosidase by cellobiose. Quantitative release of p-nitrophenol, determined under alkaline conditions, is a measure for enzymatic activity. In the incubation 90 microL substrate solution reacts with 10 microL sample solution containing between 1 and 6 AGI per ml. After 425 seconds of incubation, the enzymatic reaction is stopped by adding 75 microL alkaline (41.0 g/l) TRIS solution. Subsequently the absorbance is measured at a wavelength of 405 nm. For this method an amyloglucosidase standard preparation from Aspergillus niger was used for calibration of the system. Accordingly, an absorbance increase of 0.4 at 405 nm corresponds to an amyloglucosidase activity of 3 AGI per ml. The measuring range of the method is from 1 to 6 AGI per ml which corresponds to an increase of absorbance between 0.14 and 0.80.

### Sugar Profile Analysis

The samples of the proline-specific protease purified by ion-exchange chromatography, were 10 times diluted in degassed beer (Heineken Pilsener, Premium Quality) and incubated overnight at 37° C. The sugar profiles of the incubated beers were analysed on a Biorad Aminex HPX42A, 300×7.8 mm column, using a Biorad cation and anion changer to remove excess salts. Maltose, maltotriose and alpha-cyclodextrine hydrate were used as molecular weight references. Quantitative information was obtained by a calibration with glucose. Column temperature was 85 degrees C. with a flow of 0.5 ml/min of MilliQ water.

### EXAMPLES

### Example 1

### The Effect of Amylolytic Side Activities on the Polysaccharide and Sugar Composition of Beer

To estimate the negative effect of contaminating amylolytic side activities, the crude A. niger derived proline-specific endoprotease preparation (cf. WO 02/046381) was added to a degassed lager beer and incubated overnight at 37° C. To illustrate the case, in this experiment an overdoses of the proteolytic enzyme (1.0 PPU/I beer whereas a dosage of 0.25 PPU/I beer would be more than sufficient for haze prevention) was used. As a reference the same buffer volume (but without any enzymatic activity) also was added to beer and also incubated overnight. The next morning both beer samples were subjected to a sugar analysis according to the procedure detailed in the Materials and Methods section. From the results obtained (see Table 1) it is obvious that as a result of the enzyme incubation with the crude preparation of the proline-specific endoprotease, the polysaccharides present in the lager beer are almost completely degraded to generate many different oligosugars as well as glucose. For example, glucose, not present in the reference beer sample, is abundantly present in the enzyme treated beer. If such a conversion would take place during the fermentation phase of beer production, the yeasts would rapidly consume all the glucose and maltose hereby generating additional ethanol. Such changes are clearly noticeable as they change the mouthfeel as well as the ethanol percentage of the final beer. This simple experiment demonstrates that undesirable, amylolytic activities are present in the crude, proline-specific endoprotease preparation.

TABLE 1

| | Crude proline-specific | |
|---|---|---|
| Sugar/polysaccharide | protease | reference |
| Glucose | 57 | Nd |
| DP2 (maltose) | 30 | 6 |
| DP3 (maltotriose) | 15 | 14 |
| DP4 (maltotetraose) | 21 | 26 |
| polysaccharides | 6 | 74 |

### Example 2

### The Chromatographic Removal of Amylolytic Side Activities from the Proline-Specific Endoprotease from Aspergillus niger

In order to remove the amylolytic side activities from the A. niger derived proline-specific endoprotease preparation detailed in WO 02/046381, a number of chromatographic resins were screened. For this purpose the cation exchanger SP Sepharose 6FF and the hydrophobic interaction (HIC) resin butyl Sepharose 6FF (Amersham Biosciences Europe) were selected. Both resins were tested in Tricorn 5/100 columns (CV=2.2 ml) using an ÄKTA Explorer 100 controlled by UNICORN 3.20 and an ÄKTA Purifier controlled by UNICORN 3.21 in combination with a FRAC-950 fraction collector. After elution all fractions generated were tested for proline-specific endoprotease activity and amylolytic activities using methods specified in the Materials and Methods section.

Using a diafiltrate of the A. niger derived proline-specific endoprotease with an enzymatic activity of 10 PPU/ml, a pH of 4 and a conductivity of about 4 mS/cm as starting material, the SP-Sepharose-6FF chromatography was conducted under the following conditions

| Buffer A | 20 mM Citrat, 0.085M NaCl, pH 3.0 |
|---|---|
| Buffer B | 20 mM Citrat, 1.0M NaCl, pH 3.0 |
| Start conc. B (%)/Start | 0/10.7 |
| cond. (mS/cm) | |
| Flow rate (ml/min) | 0.48 |
| Sample volume (ml) | 0.40 |
| Wash volume (CV) | 6.1 |
| Flow through and wash | 1.0 and 11.0 |
| fraction sizes (ml) | |
| Gradient | 0-40% B in 10 CV; 100% for 3 CV |
| Eluate fraction size (ml) | 1.0 |

Under the chromatography conditions as used, the protease was bound to the resin whereas the main contaminating amylolytic activities showed no binding. The SP Sepharose chromatography showed an acceptable separation of proteolytic and amylolytic activities even though the isoelectric points of the protease and the amylolytic activities were found to be less than 0.8 pH units apart.

The HIC chromatography was conducted under the following conditions. Also, here a diafiltrate of the A. niger derived proline-specific endoprotease having an activity of 10 PPU/ml and a pH of 4 was used as the starting material. This diafiltrate was diluted two times with 20 mM citrate buffer containing 2 M Na₂SO₄ (pH 4.2, G=121 mS/cm) and was subsequently sterilized by filtration (0.2 μm) before loading on the column.

| | |
|---|---|
| Resin | Butyl Sepharose 6 FF |
| Column type | XK26 |
| Column volume (ml) | 107 |
| Buffer A | 20 mM citrate + 1M Na₂SO₄ |
| | (pH 4.2; G = 94 mS/cm) |
| Buffer B | 20 mM citrate + 0.02M Na₂SO₄ |
| | (pH 4.2; G = 6 mS/cm) |
| Flow rate (ml/min) | 15 (or 170 cm/h) |
| Equilibration | 0 or 20% buffer B (94 or 82 mS/cm) |
| Sample volume (ml) | 76-77 ml (with 1 M Na₂SO₄ as |
| | end concentration) |
| Wash | 20% buffer B (83 mS/cm) for 24 CV |
| Flow through and | 38.5 ml and collection of total |
| wash fraction sizes (ml) | wash volume or total selection of |
| | flow through and wash |
| Elution (step) | 100% buffer B for 12 or 15 CV |
| Eluate fraction size (ml) | 10 or 50 ml |

As the result of a considerable tailing after loading of the enzyme on the column, a long washing procedure was required to obtain baseline separation. Finally, the proline-specific endoprotease could be eluted from the column with buffer B. After pooling of the fractions containing the proline-specific proteolytic activity and measuring the remaining amylolytic side-activities, the data summarized in Table 2 were obtained. Though diluted, the purified material showed significantly lowered amylolytic side-activities, also if calculated back to the original proteolytic activity (see figures in brackets).

TABLE 2

| | | Fungal | Amylo | Proline- |
|---|---|---|---|---|
| | Amylase | amylase | glucosidase** | spec. prot. |
| Sample | (RAU/ml) | (FAU/ml) | (AGI/ml) | (PPU/ml) |
| Crude enzyme | 14 | 59 | 21 | 10 |
| After | 0.003 (0.03) | 0.14 (1.25) | <0.2 (<1.79)) | 1.12 (10) |
| purification | | | | |

### Example 3

### The Purified Proline-Specific Endoprotease from A. Niger Leaves the Maltodextrin Composition of Beer Unaffected

To test the performance of the chromatographically purified proline-specific endoprotease in beer, the experiment as described in Example 1 was repeated. However, in this case more realistic enzyme dosages were used i.e. the crude as well as the purified enzyme was dosed in an activity of 0.25 PPU/I beer. Again buffer without enzymatic activity was used as a blanc. After another incubation at 37° C. overnight, again the sugar profiles in the various beer samples were measured. The data obtained (Table 3) illustrate that the sugar profile in beer incubated with the purified enzyme is identical to the sugar profile in the reference beer. The sugar profile of the beer incubated with the crude enzyme (but this time at a realistic concentration), differs only slightly but significantly from the reference material because of its higher content of DP2 and DP3 residues. Please note that under realistic beer application conditions, the enzyme will be present during the whole fermentation and maturation process, so that even minor amylolytic contaminations will become noticable.

TABLE 3

| | | Crude | Purified |
|---|---|---|---|
| | | proline-specific | proline-specific |
| Sugar/polysaccharide | reference | protease | protease |
| Glucose | N.D. | N.D. | N.D. |
| DP2 | 6 | 11 | 8 |
| DP3 | 13 | 20 | 14 |
| DP4 | 26 | 27 | 27 |
| Polysaccharide | 80 | 79 | 81 |

1. A process for the production of a proline-specific protease preparation which is substantially free from contaminating amylolytic side activities comprising purification of a crude proline-specific protease preparation using liquid chromatography.

2. A proline-specific protease preparation which is substantially free from contaminating amylolytic side activities obtainable by the process of claim 1.

3. Use of a proline-specific protease preparation which is substantially free from contaminating amylolytic side activities for the preparation of beverages.

4. Use of a proline-specific protease preparation which is substantially free from contaminating amylolytic side activities for the preparation of protein hydrolysates.

5. Use of a proline-specific protease preparation which is substantially free from contaminating amylolytic side activities to increase the tolerance for toxic wheat gluten epitopes.
